(19) United States
(12) Patent Application Publication
(10) Pub. No.: US 2018/0168258 A1
(43) Pub. Date: Jun. 21, 2018

(54) FABRIC CONSTRUCTION WITH IMBEDDED FILTER

(71) Applicant: Petrichor SA, LLC., Palmetto, GA (US)

(72) Inventors: Peter Carlton Solle, Palmetto, GA (US); Hazel Solle, Palmetto, GA (US)

(73) Assignee: Petrichor SA, LLC., Palmetto, GA (US)

(21) Appl. No.: 15/844,104

(22) Filed: Dec. 15, 2017

Related U.S. Application Data
(60) Provisional application No. 62/435,721, filed on Dec. 17, 2016.

(51) Int. Cl. A41D 31/02; B01D 46/00; B01D 39/08; A41D 1/04; A41D 23/00; A41D 27/00; A41D 15/04; A42B 1/00; A42B 1/04; A41B 15/00; A62B 18/02; A62B 23/00; A61L 9/16; A01N 55/00

(52) U.S. Cl. CPC A41D 31/02; A41D 20/00; B01D 39/08; A41D 1/04; A41D 23/00; A41D 27/00; A41D 15/04; A42B 1/006; A42B 1/041; A41B 15/00; A62B 18/02; A62B 23/00; A61L 9/16; A01N 55/00; B01D 46/0028

(57) ABSTRACT

A garment which can be used as a face covering to reduce contamination by airborne contaminants comprises a first breathable fabric layer, a layer of flexible filter material and a second layer such that said layer of filter material in completely enclosed within said garment and positional over the users face.

This application claims priority from U.S. Provisional Patent application No. 62/435,721 filed Dec. 17, 2016 which is incorporated herein by reference in its entirety.

### BACKGROUND

The present invention relates to health concerns in areas where pollution or other airborne contamination or disease vectors may be present. More particularly, it is well known that certain parts of the world suffer from excessive industrial discharge into the atmosphere creating significant pollution concerns. Likewise there are numerous areas of the world where sanitation is so lacking that there is significant concern about the spread of contagion through the air. Indeed, the World Health Organization reported that in 2016 around 9 million people died—one in eight of total global deaths—as a result of air pollution exposure. UNICEF has been reported to have said that over 2 billion kids around the world are breathing toxic air and nobody is talking about it. Vehicle emissions, fossil fuel use, burning trash and dust all combine to make a dangerous chemical soup that leaves millions of people at risk for respiratory disease.

### SUMMARY OF THE INVENTION

A general object of the invention is to enable a user to obtain the protection of a personal air filter without giving the appearance of being sick or paranoid regarding the air quality.

In one embodiment, the product is configured as a neck scarf;

In another embodiment, the product is configured as a bandana;

In another embodiment, the product is configured as a blanket, such as a baby blanket;

In accordance with another embodiment, the product is configured as a loop scarf.

In yet another embodiment the product may be incorporated to a hoodie.

### BRIEF DESCRIPTION OF THE DRAWINGS

Referring to the drawings which are appended hereto and which form a portion of this disclosure, it may be seen that:

FIG. 1 is an exploded view of a fabric filter construction used in the various embodiments;

FIG. 2 is side profile view of a tubular embodiment:

FIG. 3 is a perspective view of an embodiment used as a cap;

FIG. 4 is a perspective view of an embodiment worn as a loop scarf;

FIG. 5A is a side elevation view of a hoodie incorporating the product;

FIG. 5B is a perspective view of the embodiment shown in FIG. 5A; and,

FIG. 6A is a side elevation view of an embodiment wherein the fabric construction is used in a collar area of a shirt or dress;

FIG. 6B is a perspective view of the embodiment wherein the fabric construction is used in a collar area of a shirt or dress; and,

FIG. 7 is a perspective view of an embodiment of the product as a head scarf.

### DETAILED DESCRIPTION

One or more of the above objects can be achieved, at least in part, by providing an attractive fabric product that can be innocuously worn and positioned over the face when the wearer desires to place a filter between the environmental air and his or her respiratory system. Although the use of conventional face masks is widespread in some countries, certain cultural and medical biases cause some people to avoid the use of such masks for their own protection or for the protection of others. I have addressed these issues by creating a product which can be inconspicuously worn and positioned to provide protection when needed.

In its broadest form my product is an air filter sandwiched between two layers of fabric. It is also possible to consider the product as a multilayer filter hybrid wherein an inner filter is sandwiched between two fabric filter layers. In an embodiment I have heretofore produced, I use a polar micro-fleece on each fabric layer and place an N95 filter in the middle using a safe fabric adhesive to sandwich and fuse the layers together. The N95 filter material is an anti-pull, anti-static, breathable, eco-friendly, mothproof, shrink-resistant, tear-resistant, water-soluble nonwoven polypropylene/polyester material having a thickness of from about 1.0 to 2.5 mm. Stitching of the layers for added durability is recommended as an additional fabrication technique and in some instances stitching may be the primary attachment for the layers. The microfleece used is made at least partially from of recycled PET bottles. However, virgin microfleece, cotton, silk, wool, linen, or any other suitable fabric may be used for the outer layers. In a subsequent embodiment, I have used stretched polytetrafluoroethylene fabric, sold under the trademark Gore-Tex®, for the outer layers. I use an industrial grade N95 filter material that can be washed and reused, however, in various applications N99, N100, R95, R99, R100, P95, P99 or P100 filter material or activated charcoal may be used.

The use of the fabric layers also facilitates the use of a reactive silane quaternary ammonium compound applied to one or both of the fabric layers form a colorless, odorless, positively charge polymer coating which chemically bonds, virtually irremovably, to the fabric layer to provide anti-microbial protection necessary to safeguard the product from microbial contamination. The fabric layers may be treated using almost any type of wet process, such as a pad or spray and the microbial may also be extruded or molded into various synthetic materials.

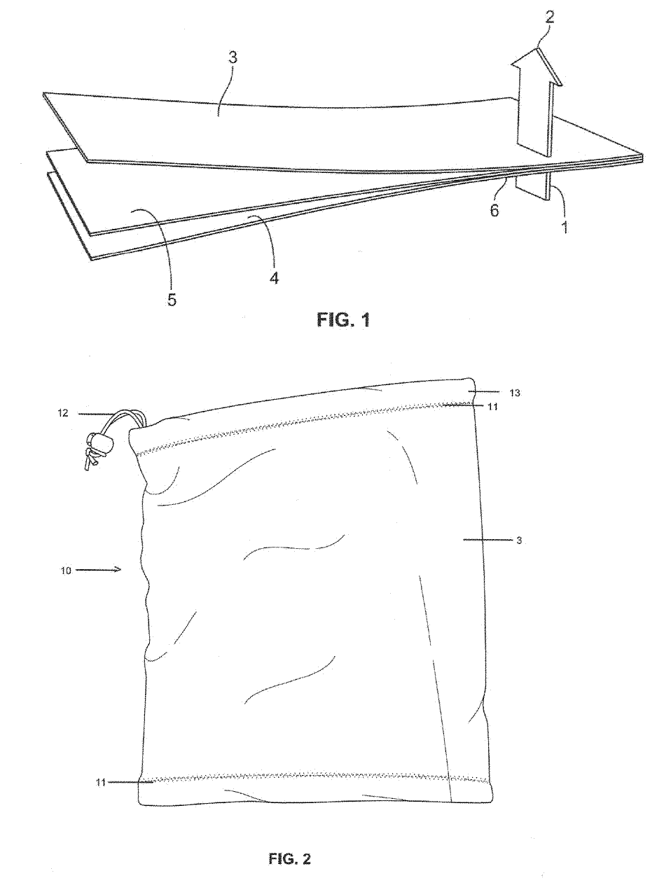

Referring to FIG. 1, it can be seen that the airflow through the product 6 in the direction from 1 to 2 passes through fabric layers 3 and 4 and filter layer 5. It should be noted that the embodiment shown in FIG. 1 is not drawn to any particular scale nor are the dimensions shown exact. It will be appreciated that the product may be configured in various ways and may be used in various ways. By way of example, the product may be used by someone using it to protect themselves from smoke inhalation during a fire, or it may be used for a baby using it at night to protect the child from germs and smoke. It should be clear that the product may be used during air travel and all other public transit, and a product using my construction may be particularly useful in areas where there are a lot of sick people on the street in cold or flu season type of thing. It is to be understood that the products made in accordance with this concept can be worn or used indoors or outdoors in as much as In certain parts of the world airborne pollution permeates the homes. The product may also be useful against household hazards including those found under the kitchen sink.

Exemplary embodiments based on the construction shown in FIG. 1, include one highly useful configuration as an elongated scarf wherein layer 5 is coextensive with the fabric layers 3 and 4 allowing the user to use any part of the scarf as a filter. In another embodiment, the filter layer 5 may be positioned near the mid-point of the scarf, or in yet another embodiment, only at one end of the scarf. In another embodiment one end of the scarf may have an activated charcoal filter and the other may have an N95 or other filter. In another form the product 6 may be configured as a bandana. As with the scarf, the bandana may have the filter layer 5 coextensive with the fabric layers 3 and 4 or the filter layer may be centered in any appropriate shape or the filter layer may be divided into one or more separate type filters. In still another embodiment, the product 6 may be configured as a baby blanket or a full size blanket. It is to be understood that the products made in accordance with this concept can be worn or used indoors or outdoors in as much as in certain parts of the world airborne pollution permeates the homes.

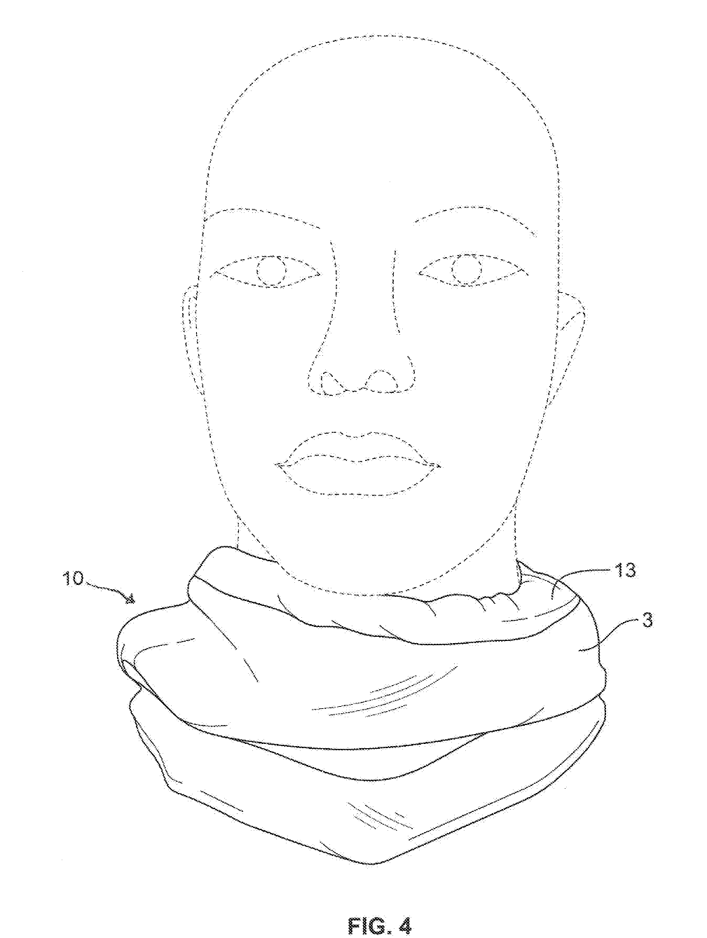

In yet another embodiment, shown in FIG. 4, the product may be configured as a convertible garment 10 in the form of a loop scarf having a coextensive filter layer, a partial filter layer, or multiple filter layers with different filters in different quadrants of the loop. In a still further application the product may configured as a modified ski-mask with the mouth and nose area of the mask including a filter layer 5. Such a mask may even be worn while sleeping in locations where airborne contaminants permeate even the living quartets.

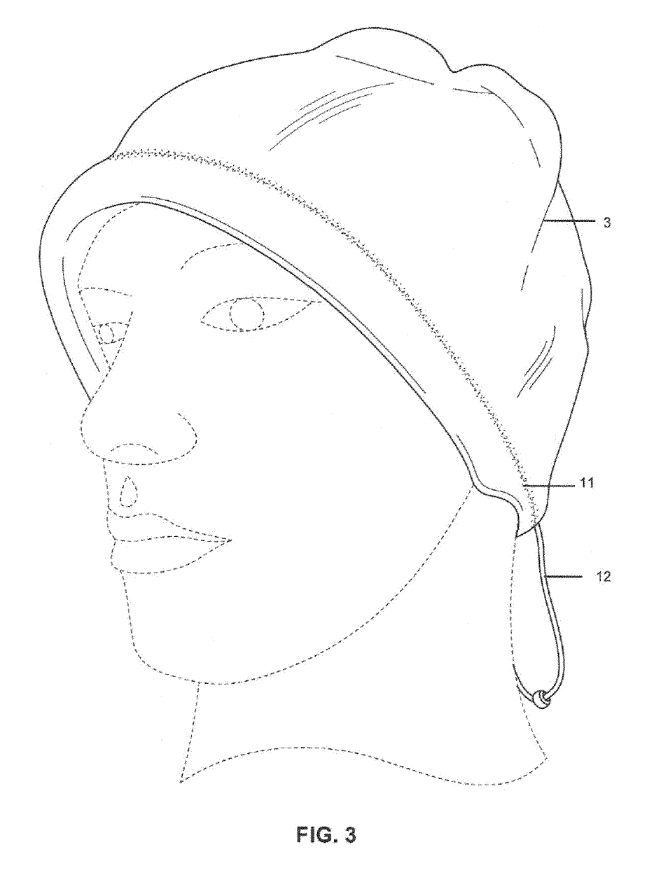

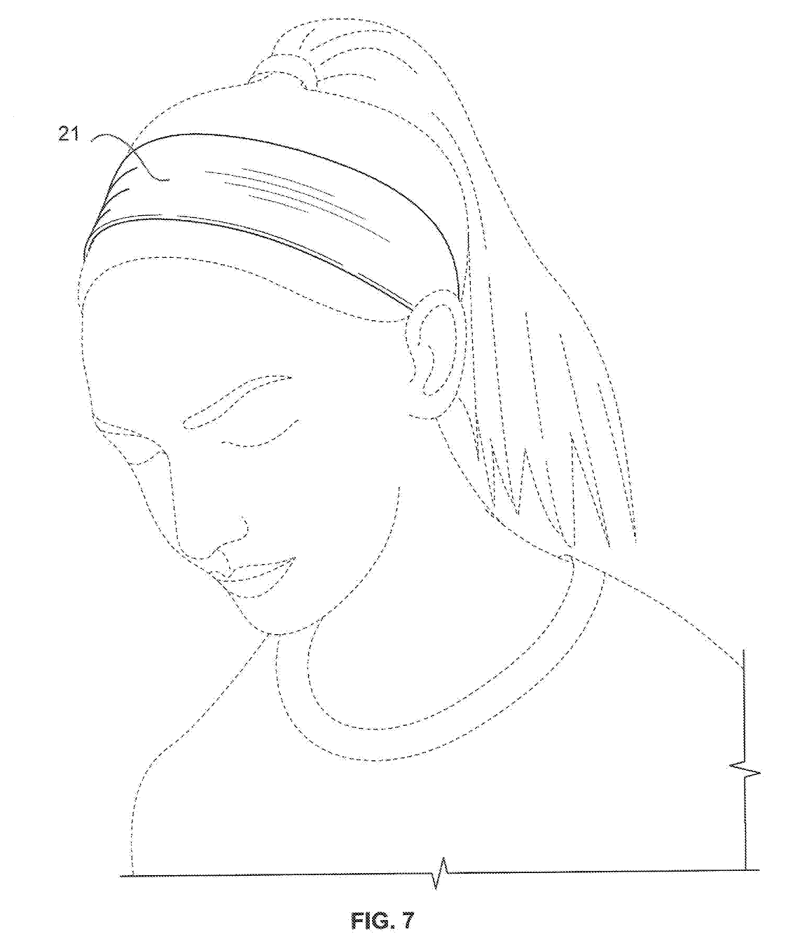

As shown in FIG. 3, the product may be incorporated in a “beanie”, a form of cap, such that the cap may be worn normally or held to one's face as a filter. Referring to FIG. 2 the convertible garment 10 embodying the current construction is shown. In FIG. 2 the product is shown sewn in a continuous loop such that only an outer layer 3 or 4 is visible. It will be appreciated that the inner and outer layer may be one piece of fabric folded over and encasing the filter layer 5, or may be a woven tube within which the filter layer 5 is disposed. In ether event in this embodiment the layers are stitched together at 11 and the stitching forms a conduit 13 for a drawstring 12. The inner diameter of the loop is sufficient to pass over a person's head such that the garment may be worn as a loop scarf. When the wearer encounters air that should be filtered, the garment can be pulled up over the nose and the drawstring 12 can be tightened around the head to hold the garment in position as a mask. Similarly, when no need for filtration exists, the drawstring may cinch up opening on one margin of the loop to form the beanie shown in FIG. 2. The garment can thus be readily disposed about the head covering the face as just described. Yet another embodiment is shown in FIG. 7 wherein the fabric construction is used in a hair band 21 that can also be disposed as a mask.

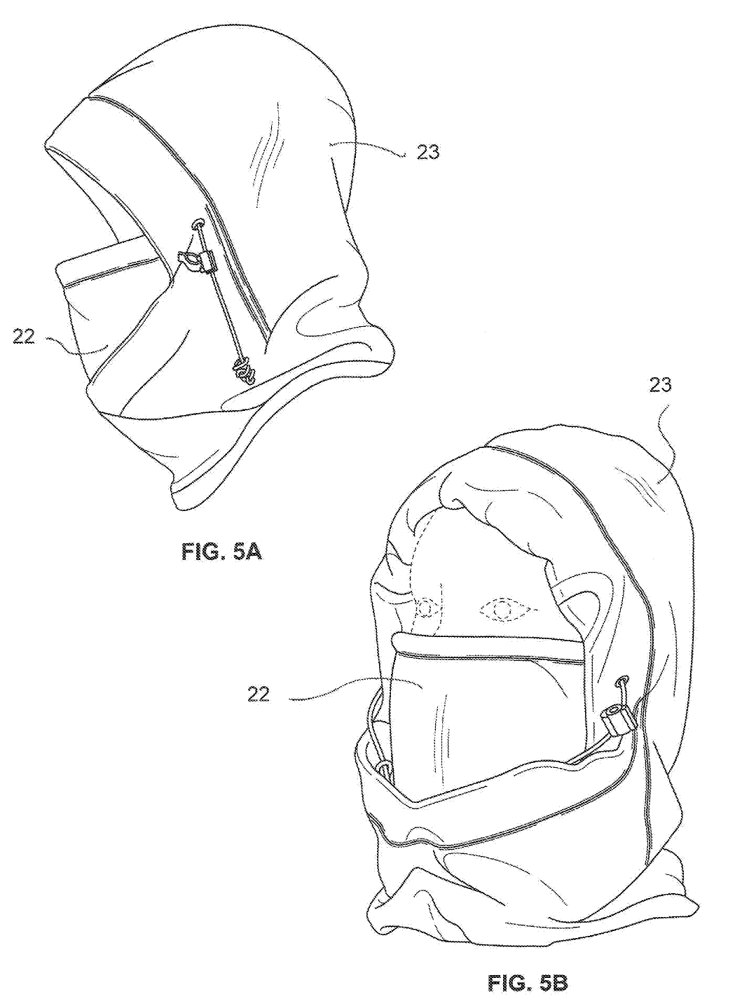

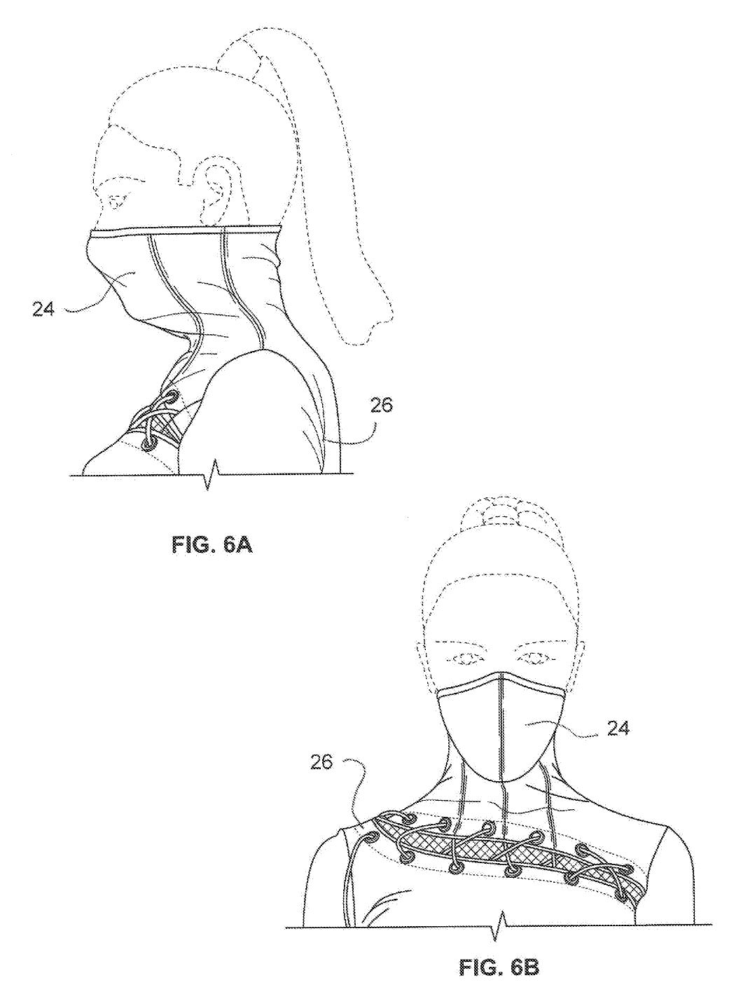

It should also be noted that the fabric construction product 6 as shown in FIG. 1 can be incorporated into a panel 22 of a hoodie 23 as shown in FIG. 5A and FIG. 5B such that the user may bring the panel across their nose and mouth when the environment indicates that the air should be filtered. Likewise the fabric construction product 6 shown in FIG. 1 can be incorporated in the neckline 24 or collar of a shirt, blouse, dress or jacket 26 as shown in FIGS. 6A and 6B so that the wearer can easily position the fabric construction across their nose and mouth. As will be understood, the product lends itself to multiple applications many of which are not listed herein.

While in the foregoing specification this invention has been described in relation to certain embodiments thereof, and many details have been nut forth for the purpose of illustration, it will be apparent to those skilled in the art that the invention is susceptible to additional embodiments and that certain of the details described herein can be varied considerably without departing from the basic principles of the invention.

What I claim is:

1. A fabric construction comprising a layer of filter material adapted to filter airborne contaminants from air passing through said layer of filter material and a first and second layer of fabric sandwiching said layer of filter material there between to define a fabric that may be disposed over said user's mouth and nose to mitigate against inhaling airborne contaminants.

2. The fabric construction as defined in claim 1 wherein the layer of filter material is a N95 filter.

3. The fabric construction as defined in claim 1 wherein the layer of filter material is substantially smaller than said first and second layers of fabric such that the layer of filter material is contained only within a predetermined area between said first and second layers.

4. The fabric construction as defined in claim 3 wherein a second layer of filter material is disposed between said first and second layers of fabric and is contained in a second predetermined area between said first and second layers of fabric.

5. The fabric construction as defined in claim 4 wherein said a layer of filter material and said second layer of filter material are selected from the group consisting of N99, N100, R95, R99, R100, P95, P99, P100 or activated charcoal filter material.

6. The fabric construction as defined in claim 5 wherein said first and second layers of fabric material are selected from the group consisting of microfleece, cotton, silk, wool, stretched polytetrafluoroethylene, polyester or linen materials.

7. The fabric construction as defined in claim 6 wherein said first and second layers of fabric material are treated with a biocide.

8. The fabric construction as defined in claim 7 wherein said biocide is a reactive silane quaternary ammonium compound.

9. The fabric construction of claim 4 wherein said first and second layers of fabric form an elongated scarf.

10. The fabric construction of claim 3 wherein said first and second layers of fabric form an article selected from the group consisting of an elongated scarf, a loop scarf, a beanie, or a hoodie.

11. The fabric construction as defined in claim 1 wherein said a layer of filter material is selected from the group consisting of N99, N100, R95, R99, R100, P95, P99, P100 or activated charcoal filter material.

12. The fabric construction as defined in claim 11 wherein said first and second layers of fabric material are selected from the group consisting of microfleece, cotton, silk, wool, stretched polytetrafluoroethylene, polyester or linen materials.

13. The fabric construction as defined in claim 12 wherein said first and second layers of fabric material are treated with a biocide.

14. The fabric construction of claim 13 wherein said first and second layers of fabric form an article selected from the group consisting of an elongated scarf, a bandana, a handkerchief, a blanket, a loop scarf, a beanie, or a hoodie.

15. The fabric construction of claim 12 wherein said first and second layers of fabric form an article selected from the group consisting of an elongated scarf, a bandana, a handkerchief, a blanket, a loop scarf, a beanie, or a hoodie.

16. The fabric construction of claim 11 wherein said first and second layers of fabric form an article selected from the group consisting of an elongated scarf, a bandana, a handkerchief, a blanket, a loop scarf, a beanie, or a hoodie.

17. The fabric construction of claim 11, wherein said first and second layers form a loop scarf having a drawstring disposed about the loop scarf to adjust the size of a portion of said loop scarf such that said loop scarf is reconfigurable as a beanie and a mask.

18. The fabric construction of claim 1 wherein said first and second layers of fabric form an article selected from the group consisting of an elongated scarf, a bandana, a handkerchief, a blanket, a loop scarf, a beanie, or a hoodie.

19. The fabric construction as defined in claim 18 wherein said first and second layers of fabric material are treated with a biocide.

20. The fabric construction as defined in claim 19 wherein said first and second layers of fabric material are selected from the group consisting of microfleece, cotton, silk, wool, stretched polytetrafluoroethylene, polyester or linen materials.
